(19) United States
(12) Patent Application Publication
(10) Pub. No.: US 2014/0194500 A1
(43) Pub. Date: Jul. 10, 2014

(54) Methods For Treating of SARS

(71) Applicants: Kookmin University Industry Academic Cooperation Foundation, Seoul (KR); Dongguk University Industry-Academic Cooperation Foundation, Seoul (KR)

(72) Inventors: Young-Sam Keum, Gyeonggi-do (KR); Yong-Joo Jeong, Gyeonggi-do (KR); Mi-Sun Yu, Gyeonggi-do (KR); Young-Won Chin, Seoul (KR)

(21) Appl. No.: 13/736,780

(22) Filed: Jan. 8, 2013

(51) Int. Cl. A61K 31/352

(52) U.S. Cl. CPC A61K 31/352
USPC 514/456

(57) ABSTRACT

Provided is a method of treating SARS in mammals. The method includes administering a therapeutically effective amount of at least one composition selected from the group consisting of myricetin, scutellarein and a pharmaceutically acceptable salt thereof to a mammal in need of treatment of SARS diseases to suppress the activities of SARS coronavirus helicase.

### BACKGROUND OF THE INVENTION

1. Field of the Invention

The present invention relates to a method of treating a severe acute respiratory syndrome (SARS) in mammals by suppressing the activities of SARS coronaviruses. More particularly, the present invention relates to a method of treating SARS using at least one ingredient selected from the group consisting of myricetin, scutellarein and a pharmaceutically acceptable salt thereof.

2. Background Art

SARS is an atypical pneumonia, primarily transmitted by respiratory droplets or personal contacts. SARS was an epidemic illness that occurred between 2002 and 2003, and caused more than 700 deaths around the world (more information can be found at http://www.who.int/csr/sars/en). Since the first diagnosis in Guangdong Province, China, successive outbreaks have occurred in 29 countries and about 20% of the patients inflicted with the SARS virus have eventually developed the symptoms of acute respiratory distress syndrome (ARDS), which required a mechanical ventilation support for survival. 50% of the patients who developed ARDS eventually died, although the mortality varied, depending on age. In addition, the rapid spreading of SARS did not allow for controlled clinical treatments during the outbreak and, therefore, empirical strategies were employed to treat patients with such agents as antiviral drugs, steroids, and type-I interferons; however, in a retrospective review of the literature, none of the medications actually benefited patients. Therefore, there is a need to develop effective anti-SARS viral agent(s) in the event of a future SARS outbreak.

SARS-CoV was isolated and shown to be a class of coronaviruses that are single stranded RNA viruses with a genome of 29,751 bases. Based on the genomic sequence, the SARS-CoV was found to be only moderately related to other human coronaviruses, HCoV-OC43 and HCoV-229E, and did not resemble any of the three previously known groups of coronaviruses (Marra, M. A. et. al, Science 2003, 300, 1399). Coronaviruses are members of a family of enveloped viruses that replicate in the cytoplasm of animal host cells. Upon infection of target cells, the genome of SARS-CoV is translated into two large replicative polyproteins that are subsequently processed into a number of non-structural proteins (nsPs) by the viral protease (Ivanov, K. A et. al, J Virol, 2004, 78, 5619). These nsPs include the RNA-dependent RNA polymerase and the helicase. Since the viral helicase is essential to viral genome replication, it is currently considered a potential target for anti-viral drug development.

### SUMMARY OF THE INVENTION

The present invention is directed to providing a method of treating SARS in mammals including administering a therapeutically effective amount of at least one composition selected from the group consisting of myricetin, scutellarein and a pharmaceutically acceptable salt thereof to a mammal in need of treatment of SARS diseases to suppress the activities of SARS coronavirus helicase.

The composition may suppress the activities of SARS coronavirus helicase nsP13.

Also, the composition may suppress the ATP hydrolysis activity of the SARS coronavirus helicase nsP13.

The mammal may be humans.

### BRIEF DESCRIPTION OF THE DRAWINGS

FIG. 1 is a schematic representation of FRET-based dsDNA unwinding assay.

FIG. 2 represents an inhibition of the dsDNA unwinding activity of the SARS CoV helicase in the presence of 10 μM natural compounds.

FIG. 3 represents an inhibition of the dsDNA unwinding activity of the HCV helicase in the presence of 10 μM natural compounds.

FIG. 4 is a schematic representation of the ATP hydrolysis assay.

FIG. 5 is represents an inhibition of the ATP hydrolysis activity of the SARS CoV helicase in the presence of 10 μM natural compounds.

FIG. 6 represents an inhibition of the ATP hydrolysis activity of the HCV helicase in the presence of 10 μM natural compounds.

FIG. 7 is a structure of myricetin and scutellarein.

FIG. 8 represents an IC₅₀ value of nsP13 ATPase activity by myricetin and scutellarein.

FIG. 9 represents the effects of myricetin and scutellarein on the growth of normal breast epithelial MCF10A cells.

### DETAILED DESCRIPTION OF THE INVENTION

The present invention is directed to providing a natural flavonoid for suppressing the activities of SARS coronaviruses. In this regard, the inventors have conducted research to find natural compounds, which effectively suppress the activities of SARS coronavirus helicase, from a total of 64 purified natural compounds.

Then, the inventors have found that, among the natural compounds, myricetin and scutellarein were potent chemical inhibitors of the SARS coronavirus helicase, which suppress the ATP hydrolysis activity of SARS coronavirus helicase nsP13.

Although the finding of an anti-viral compound has been delayed due to the risk of handling living viruses, the present invention is related to a simple cell-free system requiring no handling of living viruses, and a screening system according to the present invention will contribute to finding a compound targeting a virus helicase in the future.

Both of the myricetin and scutellarein are flavonoid-based compounds naturally occurring in all the medicinal plants, and serve to strongly suppress the SARS coronavirus activities by exerting influence on the ATP hydrolysis activity.

The present invention includes the myricetin and scutellarein for suppressing the activities of SARS coronaviruses, and a pharmaceutically acceptable salt thereof. An acid addition salt formed from a pharmaceutically acceptable free acid may be used as the pharmaceutically acceptable salt. The acid addition salt may be obtained from an inorganic acid such as hydrochloric acid, nitric acid, phosphoric acid, sulfuric acid, hydrobromic acid, hydriodic acid, sulfurous acid or phosphorous acid, or a non-toxic organic acid such as an aliphatic mono- and di-carboxylate, a phenyl-substituted alkanoate, hydroxy alkanoate, alkanedioate, an aromatic acid, or an aliphatic and aromatic sulfonic acid. Such a pharmaceutically non-toxic salt may include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartarate, methanesulfonate, propanesulfonate, naphthalene-1-sulfonate, naphthalene-2-sulfonate or mandelate.

Also, the present invention is directed to providing a pharmaceutical composition for treating or preventing SARS, which includes at least one selected from the group consisting of a flavonoid selected from myricetin and scutellarein, and a pharmaceutically acceptable salt thereof as an effective ingredient.

The myricetin and scutellarein and the pharmaceutically acceptable salt thereof may be administered alone to a mammal, and may also be administered in combination.

The pharmaceutical composition for treating or preventing SARS according to the present invention includes the flavonoid as the effective ingredient at 0.1 to 99% by weight, based on the total weight of the composition.

The pharmaceutical composition may be effectively used to treat or prevent symptoms such as a high fever caused by infection of SARS coronaviruses by suppressing the ATP hydrolysis activity of SARS coronavirus helicase nsP13 to inhibit the growth of the SARS coronaviruses.

The term “pharmaceutical composition” used herein means a preparation including a chemical ingredient such as a physiologically suitable carrier and excipient and at least one of the effective ingredients as described above. The term “effective ingredient” used herein means a preparation having biological effects. The term “physiologically acceptable carrier” or “pharmaceutically acceptable carrier” means a carrier or diluent that does not give a stimulus to an organism and destroy the natures and bioactivities of an administered compound. A lubricant, a disintegrating agent, a solubilizing agent, a dispersing agent, a stabilizing agent, a suspending agent, a coloring agent, or a flavoring agent may be used as the carrier, and a buffering agent, a preservative, a painkilling agent, a solubilizing agent, an isotonic agent, or a stabilizing agent may be used as an injectable agent.

In addition, the composition according to the present invention may be properly administered using a sustained-release system. Exemplary examples of sustained-release compositions may include a semi-permeable polymer matrix in a molded product such as a film or a microcapsule. The sustained-release matrix may include polylactide, a copolymer of L-glutamic acid and γ-ethyl-L-glutamate, poly(2-hydroxyethyl-methacrylate), ethylenevinylacetate, or poly-D-(−)-hydroxybutyric acid.

A sustained-release formulation including the composition according to the present invention includes a liposome-entrapped composition according to the present invention.

For parenteral administration, in one exemplary embodiment, the composition according to the present invention may be formulated by being mixed with a pharmaceutically acceptable carrier, that is, a carrier that is not toxic to a blood receiver at the dose and concentration used and has compatibility with other ingredients of a formulation, at a unit dose in an injectable form (a solution, a suspension or an emulsion). Preferably, the formulation does not include another compound known to be toxic to an oxidizing agent and a polypeptide. In general, the formulation is prepared by bringing the composition according to the present invention into uniform and close contact with one or both of a liquid carrier and a finely ground solid carrier. Next, a product is formed in a desired shape, as necessary. Preferably, the carrier is a parenteral carrier, and more preferably a solution that is isotonic to blood of a blood receiver. By way of example, the carrier may include water, saline, a Ringer's solution, a dextrose solution, etc. Of course, a non-aqueous vehicle such as a fixed oil and ethyl oleate may be effectively used as the liposome.

Desirably, the carrier includes a small amount of an additive such as a material for improving the isotonic and chemical stability. Such a material is not toxic to a blood receiver at the dose and concentration used and may include a buffering agent such as phosphate, citrate, succinate, acetic acid, another organic acid, or a slat thereof; an antioxidant such as ascorbic acid; a low molecular weight polypeptide (having less than approximately 10 residues) (e.g., polyarginine or tripeptide); a protein such as serum albumin, gelatin or immunoglobulin; a hydrophilic polymer such as polyvinylpyrrolidone; an amino acid such as glycine, glutamic acid, or aspartic acid; a monosaccharide, a disaccharide, a cellulose or a derivative thereof, or a carbohydrate containing glucose, mannose or dextrin; a chelating agent such as EDTA; a sugar alcohol such as mannitol or sorbitol; a counterion such as sodium; and/or a non-ionic surfactant such as a polysorbate, a poloxmer or PEG. The pharmaceutical composition according to the present invention is typically formulated in such a vehicle according to the clinically-related/acceptable protocol. It could be seen that the above-described excipient, carrier or stabilizing agent may be used to prepare a salt of the composition according to the present invention.

Also, the composition of the present invention is properly formulated in an acceptable carrier vehicle to prepare a pharmaceutical formulation, preferably a cell-free formulation. According to one exemplary embodiment, the buffering agent used in the formulation may be used immediately after mixture, or stored for use in the future. When the buffering agent is used immediately, the composition of the present invention may be formulated at a proper pH value using mannitol, glycine and phosphate. When the resulting mixture is stored, the composition of the present invention may be formulated in the buffering agent at a proper pH value in the presence of a surfactant that serves optionally to enhance solubility of the composition according to the present invention at this pH value. A final preparation may be in a sable liquid phase or a lyophilized solid phase.

The composition according to the present invention may be formulated using any method suitable for administration. Here, a desirable formulation include approximately 2 to 20 mg/ml of the composition of the present invention, approximately 2 to 50 mg/ml of an osmolyte, approximately 1 to 15 mg/ml of a stabilizing agent, and a buffer at pH approximately 5 to 6, preferably pH approximately 5 to 5.5. Preferably, the osmolyte is an inorganic salt at concentration of approximately 2 to 10 mg/ml, the sugar alcohol is in a range of approximately 40 to 50 mg/ml, the stabilizing agent is one or both of benzyl alcohol and phenol, and the buffer is an acetate buffer.

For clinical administration, the pharmaceutical composition of the present invention may be administered orally or parenterally, for example, be applied intravenously, subcutaneously, intraperitoneally or locally.

That is, the pharmaceutical composition of the present invention may be prepared into a formulation for oral administration, for example, a tablet, troches, lozenge, a water-soluble or oily suspension, a processed powder or a granule, an emulsion, a hard or soft capsule, syrup or an elixir. To prepare the formulations such as a tablet and a capsule, the pharmaceutical composition include a binder such as lactose, saccharose, sorbitol, mannitol, starch, amilopetine, cellulose or gelatin; an excipient such as calcium diphosphate; a disintegrating agent such as corn starch or sweet potato starch; or a lubricant such as magnesium stearate, calcium stearate, sodium stearyl fumarate or polyethylene glycol wax. The capsule formulation may include a liquid carrier such as fatty oil in addition to the above-described ingredients.

Also, the pharmaceutical composition of the present invention may be administered parenterally through subcutaneous, intravenous, intramuscular or intrathoracical injection. To prepare a formulation for parenteral administration, the flavonoid was mixed in water with a stabilizing agent or a buffering agent to prepare a solution or suspension, which was put into an ampule or a vial to prepare a formulation for parenteral administration in a unit administration form.

Typically, the effective ingredient may be, for example, present at an amount of 0.1 to 99% by weight, preferably 0.5 to 50% by weight of the pharmaceutical formulation.

A preferred dose of at least one ingredient selected from the group consisting of myricetin, scutellarein and a pharmaceutically acceptable salt according to the present invention varies according to the condition and weight of a patient, the severity of a disease, the type of a drug, and a route and duration for administration, but may be properly selected by those skilled in the related art. To realize the desirable effects, however, the ingredient of the present invention may be administered daily at a dose of 0.0001 to 100 mg/kg, preferably 0.001 to 100 mg/kg. The administration may be performed once a day or in divided doses a day. In any terms, the dose is not intended to limit the scope of the present invention.

Hereinafter, a method of purifying a protein and a nucleic acid and screening natural compounds that suppress more than 40% of the activities of SARS coronavirus helicase using analyses such as dsDNA unwinding and ATP hydrolysis will be described in detail.

### 1. Purification of SARS Coronavirus Helicase

SARS coronavirus helicase was expressed in E. coli Rosetta (a protein-expressing cell), and purified as follows.

Pre-cultured cells were inoculated in a liquid medium supplemented with kanamycin and chloramphenicol, and grown at 37° C. and 220 rpm until an OD value reached 0.6. When the culturing was completed, isopropyl f3-D-1-thiogalactopyranoside (IPTG) was added at a final concentration of 1 mM. Thereafter, a protein was overexpressed overnight at 18° C. and 150 rpm. The next day after the culturing, the resulting culture medium was centrifuged at 4° C. and 5,000 rpm for 20 minutes to collect cells. The cells were suspended in a buffer supplemented with phenylmethanesulfonylfluoride (PMSF) and a protease inhibitor cocktail, and homogenized using ultrasonic waves. The homogenized cells were centrifuged at 4° C. and 12,000 rpm for 30 minutes, and only a supernatant was collected and run through a nickel column at a rate of 0.5 ml/min. Then, the cells were washed with a volume of a buffer 50 times higher than that of the column, and cell fractions were eluted at an increasing concentration of 50 mM imidazole (low concentration) to 250 mM imidazole (high concentration). The cell fractions were confirmed on 10% SDS-PAGE. Then, clean samples having a molecular weight (approximately 70 kDa) corresponding to that of SARS coronavirus helicase were collected, and the next column was prepared.

A collection of the samples was concentrated to 8 mL using an ultrafiltration system, and run through a gel filtration column (Sigma, Sephadex G-100) at a rate of 0.3 ml/min. Proteins were separated using a difference in size by allowing a buffer to flow through the gel filtration column. The eluted proteins were confirmed on 10% SDS-PAGE. Then, clean samples having a molecular weight (approximately 70 kDa) corresponding to that of SARS coronavirus helicase were collected, mixed with a buffer for storage, and then stored at −80° C. in a freezer.

2. Purification of NS3h that is a Part of Hepatitis C Virus (HCV)-Derived Helicase

Next, NS3h that is a part of HCV-derived helicase was expressed in E. coli BL21(DE3), and purified as follows.

Pre-cultured cells were inoculated in a liquid medium supplemented with ampicillin, and grown at 37° C. and 220 rpm until an OD value reached 0.6. When the culturing was completed, IPTG was added at a final concentration of 0.5 mM, and a protein was then overexpressed for 2 hours. The culture medium was centrifuged at 4° C. and 5,000 rpm for 20 minutes to collect cells. The collected cells were suspended in a buffer supplemented with PMSF and a protease inhibitor cocktail, and homogenized using lysozyme and ultrasonic waves. The homogenized cells were centrifuged at 4° C. and 12,000 rpm for 30 minutes, and only a supernatant was collected and run through a nickel column at a rate of 0.5 ml/min. Then, the cells were sufficiently washed with a buffer, and 400 ml of cell fractions were eluted at an increasing concentration of 5 mM imidazole (low concentration) to 300 mM imidazole (high concentration). Among these fractions, the second peak generally corresponded to an NS3h protein. The fractions were confirmed on 10% SDS-PAGE, clean samples having a molecular weight (approximately 54 kDa) corresponding to that of NS3h protein were collected, and proteins were precipitated from the samples using ammonium sulfate fractionation. After centrifugation, the precipitated proteins were dissolved in 10 ml of a Q loading buffer and run through a dialysis membrane. The proteins were desalinated using 1 L of a Q loading buffer. The desalinated proteins were centrifuged, and then run through a Q sepharose column at a rate of 0.5 ml/min. Then, the proteins were washed with 20 ml of a Q loading buffer. 400 ml of protein fractions were eluted at an increasing concentration of 0 M NaCl to 0.5 M NaCl. The protein fractions were confirmed on 10% SDS-PAGE, clean samples having a molecular weight (approximately 54 kDa) corresponding to that of NS3h protein were collected, and proteins were precipitated from the samples using ammonium sulfate fractionation. The precipitated proteins were dissolved in 3 ml of a sample buffer (50 mM MOPS-Na, 10 mM NaCl, 10 mM DTT, 1 mM EDTA, pH 7.0), and dialyzed overnight using the same buffer supplemented with 15% glycerol. 100 μl of a solution of purified NS3h protein obtained by centrifugation was divided into tubes, which were stored at −80° C. in a freezer.

### 3. DNAs

Synthetic DNAs engrafted with carboxytetramethylrhodamine (TAMRA) and fluorescein were purchased from DNA Technology (Coralville, Iowa), and their concentrations were determined using their absorbance at 260 nm and absorption coefficients.

Base sequences of synthetic DNAs engrafted with TAMRA, BHQ and fluorescein were set forth in 5′-20T25Tam (5′-T₂₀GAGCGGATTACTATACTACATTAGA(TAMRA)-3′), 5′-20T25BHQ (5′-T₂₀GAGCGGATTACTATACTACATTAGA(BHQ)-3′), and 3′-0T25Flu (5′-(fluorescein)TCTAATGTAGTATAGTAATCCGCTC-3′) and 3′-15T25Flu (5′-(fluorescein)TCTAATGTAGTATAGTAATCCGCTCT₁₅-3′), respectively.

As a substrate of SARS coronavirus helicase, dsDNA obtained by reacting 15 μM 5′-20T25Tam with 10 μM 3′-0T25Flu in a 20 mM HEPES (pH7.4) buffer at 95° C. for 5 minutes and then slowly cooling the resulting reaction solution to bind two strands to each other was used. Also, as a substrate of HCV NS3h, dsDNA obtained by reacting 15 μM 5′-20T25BHQ with 10 μM 3′-15T25Flu in a 50 mM MOPS-Na (pH7.0) buffer at 95° C. for 5 minutes and then slowly cooling the resulting reaction solution to bind two strands to each other was used. 5′-20T25Tam, 5′-20T25BHQ and 3′-15T25Flu were designed to overhang 20 dT bases from the 5′ terminus and 15 dT bases from the 3′ terminus so as to load the SARS coronavirus helicase and HCV NS3h, respectively.

### 4. DNA Unwinding and ATP Hydrolysis Analysis

The dsDNA unwinding activity of SARS coronavirus helicase was tested as follows. 64 natural compounds were added to a 96-well plate at a final concentration of 10 μM. Then, the SARS coronavirus helicase was diluted with a buffer, and added to the 96-well plate at a concentration of 200 nM per well. The resulting mixture was reacted at room temperature for 10 minutes while stifling. A reaction solution including 9 mM ATP, 5 mM DTT, 20 nM dsDNA and 1 mM MgCl₂ was finally prepared, added to the reaction mixture, and reacted at 37° C. for 10 minutes. A reaction termination solution including 50 mM EDTA and 200 nM Trap DNA was finally prepared, and added to the reaction mixture. Then, a degree of emission of FAM was measured using a filter capable of emitting light of 485 nm and detecting light of 535 nm, and then digitalized. Then, the natural compounds suppressing more than 40% of the SARS coronavirus helicase activities were screened.

The ATP hydrolysis activity of SARS coronavirus helicase was tested as follows. 64 natural compounds were added to a 96-well plate at a final concentration of 10 μM. Then, the SARS coronavirus helicase was diluted with a buffer, and added to the 96-well plate at a concentration of 400 nM per well. The resulting mixture was reacted at room temperature for 10 minutes while stirring. A reaction solution including 50 mM NaCl, 2 mM ATP, 2 nM M13 (ssDNA) and 5 mM MgCl₂ was finally prepared, added to the reaction mixture, and reacted at 37° C. for 10 minutes. A chromogenic reagent composed of Malachite Green and ammonium molybdate was added to stop the reaction, and a degree of chromogenicity by formed Pi was digitalized using the absorbance measured at 620 nm. The natural compounds suppressing more than 40% of the activities of SARS coronavirus helicase were screened, and tested at increasing concentrations (0.01 μM, 0.1 μM, 0.3 μM, 0.5 μM, 0.7 μM, 1 μM, 3 μM, 5 μM, 7 μM, 10 μM, and 20 μM).

An inhibitory concentration (IC₅₀) of each of the natural compounds when each compound suppressed 50% of the SARS coronavirus helicase activities was calculated using a SigmaPlot program. The dsDNA unwinding activity of HCV NS3h was tested as follows. 64 natural compounds were added to a 96-well plate at a final concentration of 10 μM. Then, the SARS coronavirus helicase was diluted with a buffer, and added to the 96-well plate at a concentration of 200 nM per well. The resulting mixture was reacted at room temperature for 10 minutes while stifling. A reaction solution including 10 mM ATP, 5 mM DTT, 20 nM dsDNA and 5 mM MgCl₂ was finally prepared, added to the reaction mixture, and reacted at 37° C. for 20 minutes. A reaction termination solution including 50 mM EDTA and 200 nM Trap DNA was finally prepared, and added to the reaction mixture. Then, a degree of emission of FAM was measured using a filter capable of emitting light of 485 nm and detecting light of 535 nm, and then digitalized. Then, the natural compounds suppressing more than 40% of the HCV NS3h activities were screened. The ATP hydrolysis activity of HCV NS3h was tested as follows. 64 natural compounds were added to a 96-well plate at a final concentration of 10 μM. Then, the SARS coronavirus helicase was diluted with a buffer, and added to the 96-well plate at a concentration of 400 nM per well. The resulting mixture was reacted at room temperature for 10 minutes while stifling. A reaction solution including 50 mM NaCl, 2 mM ATP, 10 nM PolyU and 5 mM MgCl₂ was finally prepared, added to the reaction mixture, and reacted at 37° C. for 10 minutes. A chromogenic reagent composed of Malachite Green and ammonium molybdate was added to stop the reaction, and a degree of chromogenicity by formed Pi was digitalized using the absorbance measured at 620 nm. The natural compounds suppressing more than 40% of the SARS coronavirus helicase activities were screened.

### Example 1

### Preparing a Compound Library

The inventors prepared for a library of compounds (Table 1), and tested effects of the compounds on the activities of SARS coronavirus helicase nsP13. Although SARS-CoV contains a RNA-dependent RNA polymerase, nsP13 has been reported to possess dsDNA unwinding activity as well as the ability to translocate along the nucleic acids by hydrolyzing ATP.

The natural compounds were directly purified from various medicinal plants or purchased from commercial vendor (Chromadex Inc.) The integrity of the individual natural compounds, directly purified from natural plants was confirmed by NMR spectroscopy. All natural compounds were dissolved in DMSO at a concentration of 10 mM as a stock solution before experiments.

TABLE 1
Natural compounds and sources

| No | Compound | Source |
|---|---|---|
| 1 | daidzin | Chromadex |
| 2 | isohesperdin | Chromadex |
| 3 | galangin | Chromadex |
| 4 | sophoricoside | Chromadex |
| 5 | isoquercetin | Chromadex |
| 6 | myricetin | Chromadex |
| 7 | myricitrin | Chromadex |
| 8 | scutellarein | Scutellaria baicalensis |
| 9 | chrysin | Chromadex |
| 10 | silymarin | Chromadex |
| 11 | icaritin | Chromadex |
| 12 | curcumin | Chromadex |
| 13 | scutellarin | Scutellaria baicalensis |
| 14 | baicatein | Scutellaria baicalensis |
| 15 | hyperoside | Chromadex |
| 16 | naringin | Chromadex |
| 17 | naringenin | Chromadex |
| 18 | amentoflavone | Chromadex |
| 19 | populretin | Chromadex |
| 20 | ican [symbol] | Chromadex |
| 21 | 8-deoxygartanin | Garcinia mangostana |
| 22 | isoliquiritigenin | Glycyrrhiza glabra |
| 23 | l-isomangostin | Garcinia mangostana |
| 24 | γ-mangostin | Garcinia mangostana |
| 25 | α-mangostin | Garcinia mangostana |
| 26 | lamberiainc acid | Thuja orientalis |
| 27 | perviridamide | Aglaia perviridis |
| 28 | 4-hydroxypyramidatine | Aglaia perviridis |
| 29 | pyramidatine | Aglaia perviridis |
| 30 | Verproside | Phseudolysimachion |
| | | longifolium |
| 31 | Isovanillyl Catalpol | Phseudolysimachion |
| | | longifolium |
| 32 | 6-O-Veratroyl Catalpol | Phseudolysimachion |
| | | longifolium |
| 33 | minecoside | Phseudolysimachion |
| | | longifolium |
| 34 | Diosmetin-7-O-Glc | Phseudolysimachion |
| | | longifolium |
| 35 | Diosmetin-7-O-Glc-Xyl | Phseudolysimachion |
| | | longifolium |
| 36 | 3β-friedelanol | Bridelia cambodiana |
| 37 | friedelin | Bridelia cambodiana |
| 38 | 24-Methylianosta-9(11), | Bridelia cambodiana |
| | 25-dien-3-one | |
| 39 | 24,24-Dimethylanosta-9(11), | Bridelia cambodiana |
| | 25-dien-3-one | |
| 40 | 24-Methyl-5a-lanosta-9(11), | Bridelia cambodiana |
| | 25-dien-3-one | |
| 41 | Betulinic acid | Bridelia cambodiana |
| 42 | α-Amyrin | Bridelia cambodiana |
| 43 | Ursolic acid | Bridelia cambodiana |
| 44 | Oleanolic acid | Bridelia cambodiana |
| 45 | Stigmasterol | Bridelia cambodiana |
| 46 | β-Sitosterol | Bridelia cambodiana |
| 47 | Daucosterol | Bridelia cambodiana |
| 48 | gypenoside XVII | Panax ginseng |
| 49 | ginsenoside Rb1 | Panax ginseng |
| 50 | imperatorin | Saposhnikovia divaricata |
| 51 | hamaudal | Saposhnikovia divaricata |
| 57 | 3-O-angeloythamaudol | Saposhnikovia divaricata |
| 53 | 5-O-Methylvisamminol | Saposhnikovia divaricata |
| 54 | Ledebouriellol | Saposhnikovia divaricata |
| 55 | galic acid | Chromadex |
| 56 | methoxyeugenol | Cinnamomum |
| | | cambodianum |
| 57 | spatulenol | Thyrsanthera |
| | | suborbicularis |
| 58 | taraxerol | Thyrsanthera |
| | | suborbicularis |
| 59 | 19-hydroxy-1(10),15-rosa | Thyrsanthera |
| | diene | suborbicularis |
| 60 | aleuritolic acid | Thyrsanthera |
| | | suborbicularis |
| 61 | Marliolide | Cinnamomum |
| | | cambodianum |
| 62 | sec-O-Glucosylhamaudol | Saposhnikovia divaricata |
| 63 | 4′-O-β-D-glucosyl-5-O- | Saposhnikovia divaricata |
| | methylvisamminol | |
| 64 | prim-O-Glucosyl [symbol] ugin | Saposhnikovia divaricata |

[symbol]

indicates data missing or illegible when filed

### Example 2

### Screening Compounds

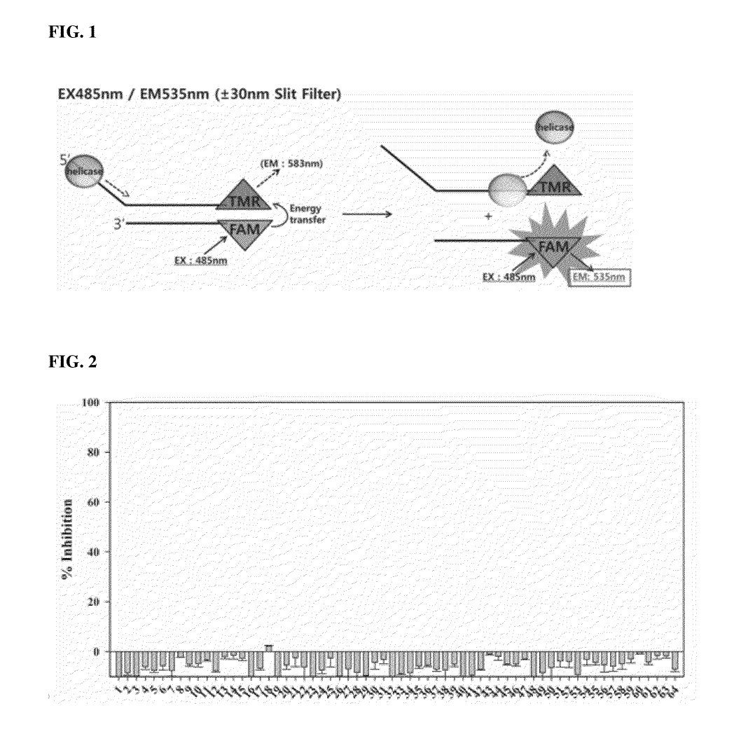

The inventors attempted to screen compounds that suppress the DNA unwinding activity of nsP13 and the dsDNA unwinding activity of nsP13 was measured using a fluorometric assay, based on the FRET (Fluorescence Resonance Energy Transfer) from the fluorescein to the carboxytetramethylrhodamine (TAMRA) (FIG. 1). FRET describes an energy transfer mechanism between two dye molecules, in which energy is transferred from a donor molecule to an acceptor molecule. This approach is highly useful in determining a dynamic interaction between two adjacent molecules. More specifically, our experimental setup was devised in such a way that FRET occurred from the fluorescein to TAMRA; thus, no fluorescence from fluorescein was generated when the two DNA strands were base-paired, but a strong fluorescence was generated and detectable due to the absence of FRET between fluorescein and TAMRA when the duplex was unwound by the nsP13 helicase.

### Example 3

### dsDNA-Unwinding Reaction

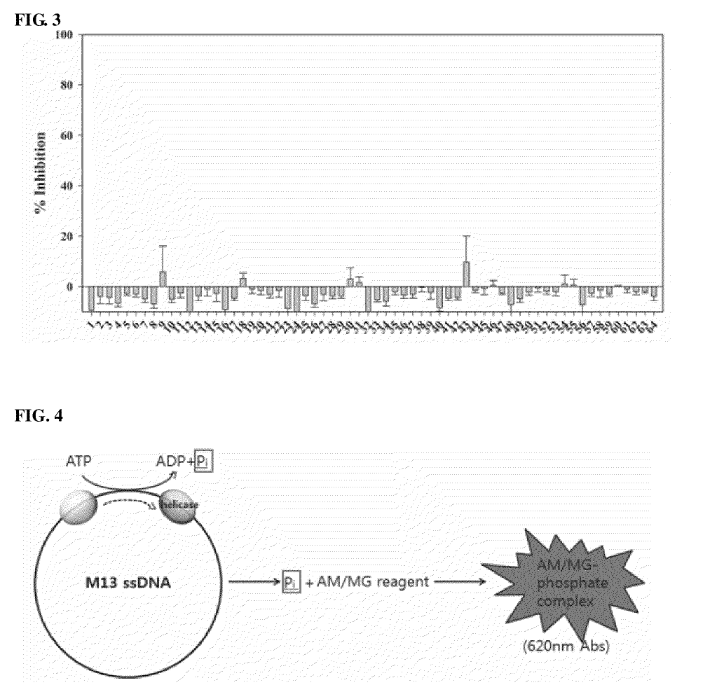

Based on this principle, we added individual natural compounds at a concentration of 10 μM to the dsDNA-unwinding reaction and measured the emitting fluorescent intensity at a wavelength of 535 nm. In these experiments, none of the natural chemicals inhibited the dsDNA-unwinding activity of SARS helicase, nsP13 (FIG. 2). In an identical experimental setup, we attempted to identify chemical inhibitors of the HCV viral helicase, NS3h, and found that none of the natural chemicals in our experiment inhibited the DNA unwinding activity of HCV viral helicase in vitro (FIG. 3).

### Example 4

### ATP Hydrolysis Assay

We then assessed whether any of these natural compounds could inhibit the ATPase activity of nsP13. ATP hydrolysis by helicases was assayed by measuring the amount of released inorganic phosphate from ATP using a colorimetric assay. Colorimetric measurements of complex formation with malachite green and molybdate (AM/MG) were performed in the presence of various concentrations of natural compounds. All experiments were repeated three times and averaged.

The ATP hydrolysis assay was conducted with nsP13 in the presence of M13 single-stranded (ss) DNA. M13 ssDNA is a 7,250 base long circular DNA that has no end and, therefore, the helicase is expected to continuously translocate along the ssDNA unless the helicase separates from the DNA. ATP hydrolysis was assessed using a colorimetric assay by measuring the release of P, through the formation of the molybdate complex (FIG. 4).

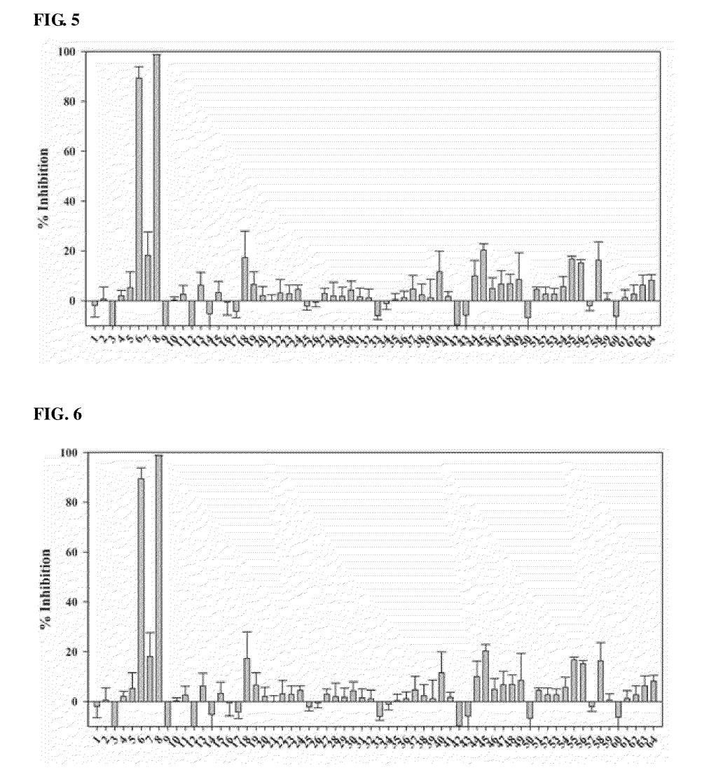

Using this experimental setup, we examined whether there were any natural compounds that inhibited the ATP hydrolysis activity of nsP13 and found that out of the 66 natural chemicals tested, myricetin (No. 6) and scutellarein (No. 8) inhibited the ATPase activity of nsP13 by more than 90% at a concentration of 10 μM, while a few compounds such as myricitrin (No. 7), amentoflavone (No. 18), diosmetin-7-O-Glc-Xyl (No. 35) and taraxerol (No. 58) exhibited some degree of inhibition (around 20%), as shown in FIG. 5. Again, we were not able to detect any compounds in our natural compound library that suppressed the ATPase activity of the HCV viral helicase (FIG. 6).

### Example 5

### Inhibitory Effects of Myricetin and Scutellarein on the ATPase Activity In Vitro

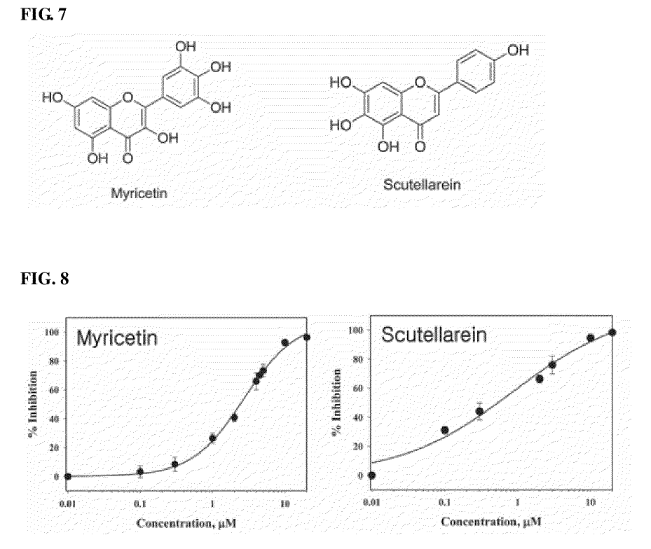

In order to determine the IC₅₀ value of 6 and 8 (FIG. 7) in suppressing nsP13 ATPase activity, we serially diluted 6 and 8 and measured their inhibitory effects on the ATPase activity of nsP13 in vitro. As a result of this analysis, IC₅₀ values of 6 and 8 were determined to be 2.71±0.19 μM and 0.86±0.48 μM, respectively (FIG. 8).

### Example 6

### Cytotoxicity

To determine whether myricetin or scutellarein possesses potential cytotoxicity in normal cells, we have exposed normal breast epithelial MCF10A cells to myricetin (2 μM) or scutellarein (2 μM) and observed whether they could exhibit inhibitory effects on the growth of MCF10A cells. Normal breast epithelial MCF10A cells were maintained in DMEM (Invitrogen, Carlsbad, Calif.) media, supplemented with 10% FBS (Invitrogen, Carlsbad, Calif.), 0.02 μg/ml epidermal growth factor (EGF), 5 μg/ml insulin, 1.25 μg/ml hydrocortisone (Sigma, St. Louis, Mo., USA) at 37° C. in 5% CO₂. MCF10A Cells were seeded in a 6-well plate at the number of 2.0×10⁵ per well and exposed to myricetin or scutellarein at the concentration. Cells were collected every 24h for 3 days and the viable cell number was calculated, using hemacytometer counting. Data are shown in mean+standard deviation and a statistical analysis was conducted with Student t-test (n=6). However, we didn't observe any statistical significance between the control group vs the myricetin or scutellarein group.

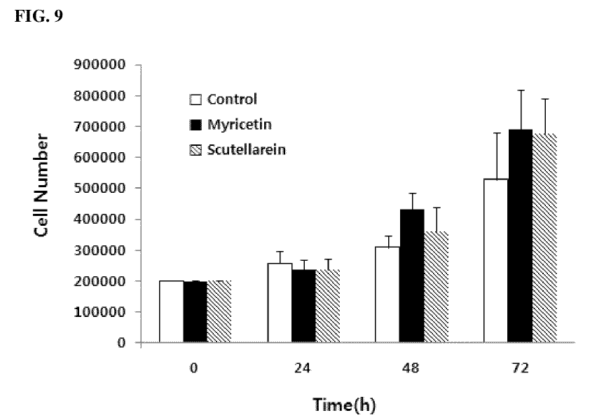

As a result, we observed that either myricetin or scutellarein did not affect the growth of MCF10A cells at cellular concentrations close to the IC₅₀ of myricetin or scutellarein (FIG. 9), suggesting that both myricetin and scutellarein are safe compounds at pharmacologically-effective concentrations.

Naturally-occurring chemicals are regarded as a great source of potential medications against various diseases. In particular, they have gained great scientific interest due to their strong neuroprotective, cardioprotective and chemopreventive activities. In the present invention, we present the evidence for the first time that myricetin and scutellarein are strong chemical inhibitors of SARS-CoV helicase and this effect is mediated through inhibition of ATPase activity, but not inhibition of helicase activity. On the other hand, myricetin and scutellarein did not suppress the helicase activity of HCV virus in our experimental setup. The reason for this discrepancy is currently unknown, but this may be due to structural difference of the ATPase domain between SARS-CoV helicase and HCV helicase. This result also indicates that suppression of SARS-CoV helicase by myricetin and scutellarein might not be mediated by affecting the protein stability and/or integrity of SARS-CoV protein in vitro, since these compounds did not seem to suppress the ATPase activity of HCV helicase protein. Collectively, we propose that myricetin and scutellarein hold a great promise for use in treating and controlling potential future SARS outbreaks; however, more preclinical/clinical studies are necessary to examine whether this effect occurs after in vivo treatment.

Hereinafter, preferred Preparative Examples of a pharmaceutical composition for suppressing the activities of SARS coronaviruses will be described. However, it should be understood that the following Preparative Examples are just examples for illustration, but are not intended to limit the scope of the present invention.

### Preparative Example 1

### Preparation of Powder

| | |
|---|---|
| Myricetin or Scutellarein | 200 mg |
| Lactose | 100 mg |
| Talc | 10 mg |

These ingredients were mixed, and the resulting mixture was filled in an airtight container to prepare a powder.

### Preparative Example 2

### Preparation of Tablet

| | |
|---|---|
| Myricetin or Scutellarein | 200 mg |
| Lactose | 100 mg |
| Starch | 100 mg |
| Magnesium stearate | 2 mg |

These ingredients were mixed, and the resulting mixture was then compressed according to a conventional method of preparing a tablet, thereby preparing a tablet.

### Preparative Example 3

### Preparation of Capsule

| | | |
|---|---|---|
| Myricetin or Scutellarein | 100 | mg |
| Crystalline cellulose | 3 | mg |
| Lactose | 14.8 | mg |
| Magnesium stearate | 2 | mg |

These ingredients were mixed, and the resulting mixture was filled in a gelatin capsule according to a conventional method of preparing a capsule, thereby preparing a capsule.

### Preparative Example 4

### Preparation of Injectable Agent

| | |
|---|---|
| Myricetin or Scutellarein | 100 mg |
| Mannitol | 180 mg |
| Sterile injectable distilled water | 2794 mg |
| Na₂HPO₄12H₂O | 26 mg |

These ingredients were put into an ampule (2 ml) at the contents as describe above according to a conventional method of preparing an injectable agent.

### Preparative Example 5

### Preparation of Solution

| | |
|---|---|
| Myricetin or Scutellarein | 100 mg |
| Isomerized sugar | 20 g |
| Mannitol | 5 g |

According to a conventional method of preparing a solution, these ingredients were added to distilled water and dissolved. Then, a proper amount of a lemon perfume was added and mixed with the ingredients, and distilled water was added to the resulting mixture, which was adjusted to a total of 100 ml. The mixture was filled in a brown vial and sterilized to prepare a solution.

What is claimed is:

1. A method of treating SARS in mammals comprising administering a therapeutically effective amount of at least one composition selected from the group consisting of myricetin, scutellarein and a pharmaceutically acceptable salt thereof to a mammal in need of treatment of SARS diseases to suppress the activities of SARS coronavirus helicase.

2. The method according to claim 1, wherein the composition suppresses the activities of SARS coronavirus helicase nsP13.

3. The method according to claim 1, wherein the composition suppresses the ATP hydrolysis activity of the SARS coronavirus helicase nsP13.

4. The method according to claim 1, wherein the mammal is humans.
